(19) United States
(12) Patent Application Publication
(10) Pub. No.: US 2024/0082267 A1
(43) Pub. Date: Mar. 14, 2024

(54) EDIBLE CBD AND/OR THC PRODUCTS AND METHODS

(71) Applicant: Innovative Patent Company, LLC, McDonough, GA (US)

(72) Inventors: Kishan Patel, Stockbridge, GA (US); Matthew Rizzio, Huntsville, AL (US); Harshad Patel, McDonough, GA (US)

(73) Assignee: Innovative Patent Company, LLC, McDonough, GA (US)

(21) Appl. No.: 17/940,716

(22) Filed: Sep. 8, 2022

(51) Int. Cl. A61K 31/00; A23G 3/42; A23G 3/48; A23G 3/54; A61K 9/00; A61K 47/26

(52) U.S. Cl. CPC A61K 31/658; A23G 3/42; A23G 3/48; A23G 3/54; A61K 9/0056; A61K 47/26

(57) ABSTRACT

An edible having health benefits of the present disclosure consists of corn syrup, cane sugar, and less than or equal to 0.30% pure THC, CBD distillate and/or CBD isolate. An edible having health benefits of the present disclosure consists of corn syrup, can sugar, and greater than 0.3% THC, CBD distillate and/or CBD isolate.

### BACKGROUND

Cannabinoid (CBD) and/or THC edibles include any type of product that can be eaten that is infused with CBD and/or THC. CBD-infused edibles are a popular means of enjoying beneficial compounds found in hemp. CBD and/or THC-infused edibles are a popular means for enjoying beneficial compounds found in hemp, Cannabis, and/or marijuana. Edibles are both delicious and highly effective. These edibles are typically made by infusing high-quality CBD oil and/or THC oil with a variety of ingredients to make an edible form of CBD and/or THC

While it may take a little longer to go into effect, CBD and/or THC edibles have a benefit over smoking Cannabis or inhaling a CBD and/or THC vapor pen. For example, the effect of the edible lasts longer. Because the edible is typically combined with other products, the CBD and/or THC is released slowly over long periods of time as the edible digests.

Also, CBD is non-psychotropic. That is, the CBD edible does not cause a “high.” Instead, the CBD edibles cause a mellow mood uplift with a sense of positive energy.

Also, THC is a psychotropic substance. That is, the THC edible does cause a “high.” The THC edibles may or may not cause an increase in appetite and may or may not cause weight gain. Also, the THC edible may be used in cancer patients during treatment.

Furthermore, CBD edibles may be successful in treating a variety of epilepsy. CBD edibles may also be beneficial for aiding a minor injury, headache, or joint pain.

### BRIEF DESCRIPTION OF THE DRAWINGS

The present disclosure can be better understood with reference to the following drawings. The elements in these drawings are not necessarily drawn to scale but are included to clearly illustrate the principles pertaining to this disclosure. Furthermore, like reference numerals designate corresponding parts throughout the several views.

FIG. 1 is a block diagram of the machines used to create pure THC, CBD isolate, and/or CBD distillate.

FIG. 2 is a flowchart depicting the architecture and functionality of creating pure THC, CBD isolate and/or CBD distillate.

### DETAILED DESCRIPTION

The present disclosure is an edible and method of making an edible containing less than or equal to 0.3% THC, greater than 0.3% THC, CBD distillate, CBD isolate, and/or other cannabinoids. To make edibles containing less than or equal to 0.3% THC, greater than 0.3% THC, CBD distillate, CBD isolate, and/or other cannabinoids the less than or equal to 0.3% THC, greater than 0.3% THC, CBD distillate, CBD isolate, or other cannabinoids is made into a crude oil. The oil is then distilled. An edible is any type of substance that can be ingested by a human being and/or animals.

In one embodiment, the edible is a CBD and/or THC gummy. A CBD and/or THC gummy is an edible comprising of gum. It has a texture or properties of gum, which are sticky and viscid. A CBD and/or THC gummy is made from oil that has been distilled.

In one embodiment, the edible is a tincture. A tincture is an oil that may be ingested and/or deposited under the tongue.

In one embodiment, the edible is a pre- or post-workout product. The pre-post-workout product may be a shake or a protein bar, for example.

In one embodiment, the edible is a CBD and/or THC baked good or fried food, e.g., CBD and/or THC cookies or CBD and/or THC brownies, chips, and cereals. In such a scenario, the distilled CBD, THC, and/or other cannabinoid oil is mixed in with the batter.

In one embodiment, the edible is a CBD-infused, THC-infused, and/or other cannabinoid-infused chocolate. For example, a chocolate bar may be CBD-infused, THC-infused, and/or other cannabinoid-infused. They may come in flavors like milk chocolate bars, white chocolate bars, orange chocolate bars, and decadent Belgian chocolate.

In one embodiment, the edible is a condiment. The condiment may include, any type of sauce, including, but not limited to ketchup, barbeque sauce, mayonnaise, salad dressing, and the like.

In one embodiment, the edible is CBD-infused, THC-infused, and/and/or other cannabinoid-infused caffeinated and/or decaffeinated beverage. The beans and/or leaves are CBD-infused, THC-infused, and/and/or other cannabinoid-infused. The CBD, THC, and and/or other cannabinoid adds an earthy tone that does not overpower the beverage.

In one embodiment, the edible is a CBD-infused, THC-infused, and/and/or other cannabinoid-infused drink. There may be CBD-infused, THC-infused, and/and/or other cannabinoid-infused water, energy drinks, fruit juices and even powdered mixes. With a CBD-infused, THC-infused, and/and/or other cannabinoid-infused drink, the effect may or may not take longer to appear, in comparison to inhalation of CBD and/or THC, but it may last significantly longer than average.

In one embodiment, the edible may be a CBD-infused, THC-infused, and/and/or other cannabinoid-infused alcoholic drink. Beer, Wine, and even spirits can be CBD-infused, THC-infused, and/and/or other cannabinoid-infused.

In one embodiment, the edible may be a seasoning. For example, CBD-infused, THC-infused, and/and/or other cannabinoid-infused salt, pepper, and the like.

In one embodiment, the edible is a meat or a fish. The meat and/or fish may be sprayed with or infused with CBD, TCH, or another cannabinoid.

In one embodiment, the edible is animal byproducts. In this regard, milk, eggs, yogurt, or the like may be infused or sprayed with CBD, THC, and/or other cannabinoids.

In one embodiment, the edibles include tablets and pills. In this regard capsules, soft gels, sprinkle capsules, traditional tablets, orally disintegrating tablets, sublingual tablets, buccal tablets, chewable tablets, and effervescent dissolving tablets may be infused with or sprayed with CBD oil, THC oil, and/or other cannabinoid oil.

In one embodiment, the edibles are snacks. The edibles may include chips, popcorn, Oreos™, cheese puffs, Skittles™, or the like.

In one embodiment, the edibles are frozen foods. For example, the edible may be ice cream infused with CBD, THC, or other cannabinoid oil.

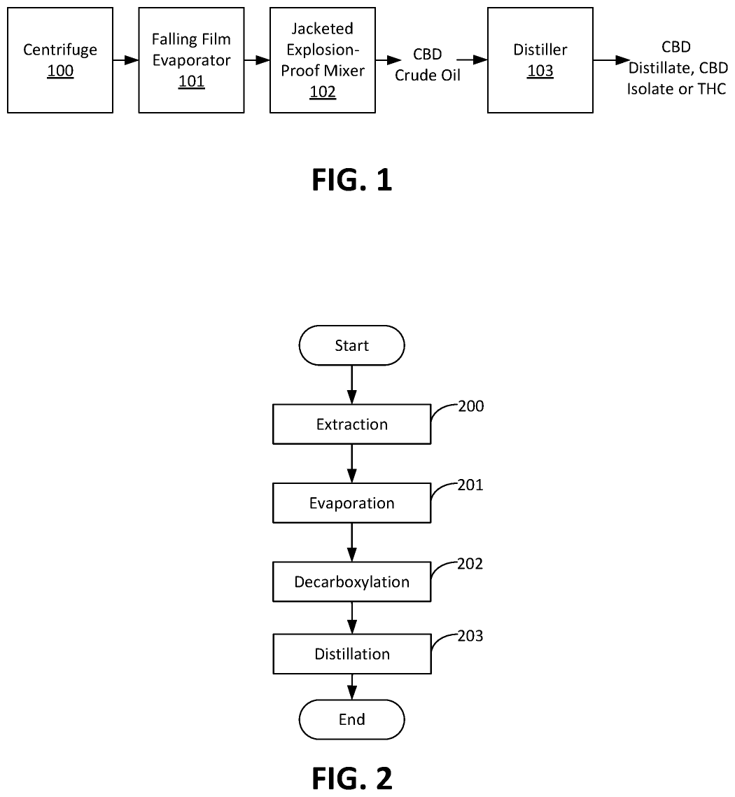

FIG. 1 is a block diagram of a system for the creation of CBD distillate, CBD isolate, and/or pure THC. CBD distillate, CBD isolate, and/or pure THC (or THC isolate) is a refined extract that has undergone the distillation process. CBD distillate, CBD isolate, and/or pure THC, which is 70%-80% THC, is a golden, viscous oil that contains around 70-80% CBD including other cannabinoids, less than 0.3% THC, or greater than 0.3 pure THC. The distillate is created by purifying a liquid by evaporation and condensation.

In addition, the process may be used to form CBD isolate, CBD distillate, or pure THC. Note that CBD isolate does not contain any other cannabinoids, terpenes, fats, lipids, or other compounds found in the hemp plant. It is a white, powdery solid that is 99+% CBD. Note that pure THC does not contain any other cannabinoids, terpenes, fats, lipids, or other compounds found in the hemp plant. It is a white, powdery solid that is 70%-80% THC.

The first block 100 is a hemp centrifuge. The hemp centrifuge is a bowl centrifuge configured to extract the hemp biomass from alcohol. In this regard, the hemp centrifuge, using over 3,000 gravitational forces (g), spins out the biomass solids from ethanol. The separated biomass and ethanol are continuously discharged from separate discharge ports.

The output from the centrifuge is fed into a falling film evaporator 101. The falling film evaporator 101 is used to recover solvent and produce high-quality Cannabis extract oil or pure THC oil. The falling film evaporator 101 acts as a solvent recovery unity to recycle high-quality solvent.

The output of the falling film evaporator 101 is fed to a jacketed explosion-proof mixing vessel 102, i.e., a double jacketed vessel. The jacketed explosion-proof mixing vessel 102 mixes liquid and/or slightly solid materials. The jacketed explosion-proof mixing vessel 102 comprises one or more paddles or blades for mixing. The jacketed explosion-proof mixing vessel 102 produces CBD crude or THC crude.

The CBD crude is a dark brown to black viscous liquid that contains the full makeup of the hemp plant, including cannabidiol, cannabichromene (CBC), cannabinol (CBN), cannabigerol (CBG), and over eighty phytocannabinoids present in the hemp plant. Notably, every compound and component of hemp is in the CBD crude. In one embodiment, THC oil crude is made from the Cannabis sativa.

The CBD crude or THC crude oil is fed to a distiller 103. The distiller 103 uses distillation to further purify the CBD crude or the THC crude oil. The distiller 103 processes the crude oil and produces a pure product. The pure product can be THC, CBD distillate or CBD isolate.

The THC, CBD distillate or CBD isolate may be used to create edibles. In this regard, the pure THC, CBD distillate, or CBD isolate may be used to make gummies, baked goods, drinks, and the like.

FIG. 2 is a flowchart that describes the creation of crude oil for use in generating distilled oil for use in edibles.

Step 200 is an extraction process. Extraction results in a liquid containing carbon dioxide and cannabinoids (CBD). To perform extraction, a centrifuge is used. Hemp or marijuana is placed within a chamber of the centrifuge. Note that hemp is any Cannabis plant, or derivative thereof that contains less than or equal to 0.3 percent delta-9 tetrahydrocannabinol (THC) on a dry-weight basis.

Also note that the extraction process may be performed with marijuana. Marijuana is a subspecies of Cannabis that has a THC content of greater than 0.3% THC on a dry weight basis.

The output liquid from the extraction system contains Ethanol. The output liquid is delivered to a chiller. The chiller circulates the liquid during chilling to decrease the time it takes to chill the liquid. At −55° the Ethanol is ready to use for extraction.

In step 201, the output from the centrifuge is evaporated. The falling film evaporator 101 is used in evaporation. The falling film evaporator is used to recover solvent and produce Cannabis extract oil. The falling film evaporator 101 acts as a solvent recovery unit to recycle high-quality solvent.

In step 202, the output from the falling film evaporator is provided to a jacketed mixer for decarboxylation. Decarboxylation activates the mixtures most essential cannabinoids, including THC and CBD. Crude CBD oil is produced after decarboxylation. In this regard, the process converts CBDa to CBD, THCa to THC, etc.

In step 203, the crude oil produced is distilled. First, any remaining ethanol is evaporated. Distillation uses heat and a vacuum to separate CBD and other cannabinoids and terpenes from less desirable compound, such as fats, waxes, and pigments. Distillation produces CBD distillate, CBD isolate, or pure THC, depending upon what the starting product was. For example, if hemp was the starting product, CBD distillate or CBD isolate is created. If the beginning product was marijuana, pure THC, THC isolate, or THC distillate is created.

There are distillation processes that may be used to distill the crude oil produced by the falling film evaporator. First, a wiped film distillation process may be used. A wiped film distillation process, also known as a thin film distillation process, purifies the temperature-sensitive crude oil. Separation is created by a turbulent liquid film at the heat transfer surface of the evaporator. The output is CBD distillate, CBD isolate, or pure THC.

A short path second pass distillation oil process may be used. This process involves the distillate traveling a short distance. Short path distillation utilizes an apparatus with a multi-position receiver and condensing head. This process is extremely limited in scale and production but can produce high-quality CBD distillate.

Crude oil is heated in a boiling flask with a magnetic stirrer. The condensing head is jacketed and requires a recirculating chiller to cool the condensing head to condense the cannabinoid vapor back into a liquid form.

The compounds are then separated through slow thermal heating with precise and controlled temperatures. As you slowly raise the boiling flask temperature, you will get multiple fractions of the distillation—the higher you go, the different compounds you will be distilling, starting with highly volatile terpenes or solvent leftover at lower temperatures. Raising the temperature will create the CBD distillate, CBD isolate, or pure THC, and the multi-position receiving flask can be adjusted to collect the different fractions into different receiving flasks.

Second pass indicates that the distillation process is controlled to bring the cannabinoid distillate away from the heavy waxes, chlorophyll, and sugars on a second pass through the distillation process.

The short path first pass distillation oil process may also be used to distill the crude oil. The short path first pass distillation oil process is like the short path second pass distillation oil process described above. However, only one distillation pass goes through the system. The first pass focuses on taking out terp strip.

In one embodiment, the CBD distillate, CBD isolate, or pure THC is used to create edibles. Note that other cannabinoids may be used to create edibles, as well. That is, the CBD distillate, CBD isolate, or pure THC is added to ingredients of edibles.

In one embodiment, the CBD distillate, CBD isolate, or pure THC is used to make gummies.

To make gummies, the following ingredients are used:
1) Corn syrup;
2) Cane sugar or other artificial sugars;
3) Fruit pectin or Gelatin.
4) Organic flavor extracts;
5) Potassium citrate;
6) Citric Acid;
7) Natural vegetable extracts for coloring;
8) CBD distillate, CBD isolate, or pure THC (≥0.3% THC or <0.3% THC).

The ingredients listed above are combined and the resulting cooking syrup is deposited in molds. The molds can be various shapes. For example, the molds may be square or a pyramid.

After depositing is complete, the molds containing the gummies are cured for forty-eight hours. During this period, cocoa butter with sugar (white chocolate) is melted, tempered, and infused with organic flavoring and powdered food coloring.

Also, during the same period, golden crystal pop rocks, or any other type of candy, are infused with moisture free powered fool coloring. The pop rocks are then cured.

Once the gummies are cured, they are placed on a conveyor-driven chocolate enrobing machine. The white chocolate is enrobed around each gummy. The pop rocks are applied by a spreader on the enrobing line.

The edible may or may not be coated by any number of ingredients. For example, marshmallows, chocolate, pretzels, any form of candy, peanuts, and the like.

The edible may also be injected. It may be fruit juice, THC, CBD isolate and CBD distillate. It might be any type of liquid.

The conveyer moves the gummies through a cooling tunnel. The conveyor moves the gummies onto a staging tables where they are packed in airtight bottles.

There are health benefits of CBD edibles. They are as follows:
1) offset anxiety and depression;
2) treat select epilepsy syndromes;
3) reduce posttraumatic stress disorder (PTSD);
4) treat opioid addition;
5) alleviate unmanageable pain;
6) ease diabetic complications; and
7) protect against neurological disease.

Furthermore, THC has health benefits as well. They are as follows:
1) reduce anxiety;
2) reduce inflammation and relieve pain;
3) control nausea and vomiting caused by cancer chemotherapy;
4) kill cancer cells and slow tumor growth;
5) relax tight muscles in people with Multiple Sclerosis (MS);
6) stimulate appetite and improve weight gain in people with cancer and AIDS.

What we claim is:

1. An edible, comprising of:
corn syrup;
cane sugar, artificial. or natural sweeteners; and
less than or equal to 0.3% pure THC, a cannabinoid distillate. cannabinoid isolate, or other cannabinoid isolates.

2. The edible of claim 1, further consisting of fruit pectin.

3. The edible of claim 1, further consisting of gelatin.

4. The edible of claim 1, further consisting of organic flavor extracts.

5. The edible of claim 1, further consisting of potassium citrate.

6. The edible of claim 1, further consisting of citric acid.

7. The edible of claim 1, further consisting of natural vegetable extracts for coloring.

8. The edible of claim 1, wherein the edible is a baked good that may or may not comprise gluten.

9. The edible of claim 1, wherein the edible is a gummy.

10. The edible of claim 1, wherein the edible is a chocolate piece.

11. The edible of claim 1, wherein the edible is an energy drink.

12. The edible of claim 1, wherein the edible is an alcoholic drink.

13. The edible of claim 1, wherein the edible is a decaffeinated or caffeinated drink.

14. The edible of claim 1, wherein the edible is a beverage.

15. The edible of claim 1, wherein the edible is a pre- and/or post-workout edible that is a shake or a protein bar.

16. The edible of claim 1, wherein the edible is a condiment.

17. The edible of claim 1, wherein the edible is a seasoning.

18. The edible of claim 1, wherein the edible is a sauce.

19. The edible of claim 1, wherein the edible is a frozen food.

20. An edible, comprising of:
corn syrup;
cane sugar, artificial sweeteners, or natural sweeteners; and
greater than 0.3% pure THC, CBD distillate, CBD isolate, or another cannabinoid isolate.

21. The edible of claim 20, further consisting of fruit pectin.

22. The edible of claim 20, further comprising gelatin.

23. The edible of claim 20, further consisting of organic flavor extracts.

24. The edible of claim 20, further consisting of potassium citrate.

25. The edible of claim 20, further consisting of citric acid.

26. The edible of claim 20, further consisting of natural vegetable extracts for coloring.

27. The edible of claim 20, wherein the edible is a baked good that may or may not contain gluten.

28. The edible of claim 20, wherein the edible is a gummy.

29. The edible of claim 20, wherein the edible is a chocolate piece.

30. The edible of claim 20, wherein the edible is an energy drink.

31. The edible of claim 20, wherein the edible is an alcoholic drink.

32. The edible of claim 20, wherein the edible is a decaffeinated or caffeinated drink.

33. The edible of claim 20, wherein the edible is beverage.

34. The edible of claim 20, wherein the edible is a pre- and/or post-workout edible that is a shake or a protein bar.

35. The edible of claim 20, wherein the edible is a condiment.
